(19) United States
(12) Patent Application Publication
(10) Pub. No.: US 2005/0078784 A1
(43) Pub. Date: Apr. 14, 2005

(54) Scanning-based detection of ionizing radiation

(76) Inventor: Tom Francke, Sollentuna (SE)

Correspondence Address: HARNESS, DICKEY & PIERCE, P.L.C., P.O. BOX 8910, RESTON, VA, 20195, US

(21) Appl. No.: 10/695,821

(22) Filed: Oct. 30, 2003

(30) Foreign Application Priority Data
Oct. 8, 2003 (SE) 0302670-5

(51) Int. Cl. G21K 1/12; H05G 1/60; G01N 23/00; A61B 6/00

(52) U.S. Cl. 378/14

(57) ABSTRACT

A scanning-based radiation detector apparatus and method are disclosed. A one-dimensional detector (16) is exposed to a fan-shaped beam (24) of ionizing radiation from a radiation source (11) as transmitted through an object, where the detector (16) is of the kind wherein charges or photons generated by interactions between the radiation beam (24) and a detection medium and traveling in a direction essentially perpendicular to the radiation beam (24), are detected. A device (17-19, 21-22) moves the detector (16) and the radiation beam (24) relative to the object while the detector (16) is arranged to repeatedly detect to thereby create an image of the object. According to the invention the detector (16) has a detecting arrangement (27, 28), which comprises a large number of individual detecting elements (27a), and means for grouping the individual detecting elements together to form a plurality of detecting stripes side-by-side, which plurality of detecting stripes are all pointing towards the radiation source (11).

### FIELD OF THE INVENTION

The invention relates generally to apparatuses and methods for scanning-based detection of radiation.

### BACKGROUND OF THE INVENTION AND RELATED ART

Various line detectors for detecting ionizing radiation are known in the art. A particular kind of line detector is the gaseous-based parallel plate detector, wherein electrons freed as a result of the interaction between an incident beam of ionizing radiation and the gas of the detector, are accelerated in a direction substantially perpendicular to the incident radiation beam and subsequently detected. This line detector is often referred to as an edge-on detector since the incident radiation beam enters the detector sideways between the plates or electrodes thereof, i.e. the radiation impinges on an edge of the detector.

The spatial resolution of such and other detectors with an edge-on incidence of radiation is often an important parameter. In many examinations it is desirable to obtain a spatial resolution, which is better than 100 microns, e.g. as good as 50 microns. This put very high demands on the detector being used—both in terms of a narrow radiation sensitive area and in terms of small detecting elements or pixels.

Therefore, the detectors are typically provided with a detecting or readout arrangement comprising a number of readout strips, which are pointing towards the used X-ray source and are broader at the far end of the detector than at the front end thereof, see e.g. U.S. Pat. No. 6,118,125 by Carlson et al. Hereby, any parallax errors, which may be introduced due to the divergence of the incident X-ray beam, are avoided.

### SUMMARY OF THE INVENTION

However, a severe drawback with the detectors using readout strips in a fan-shaped configuration described above is that each detector can be designed for one distance only between the radiation source and the detector. Such detector provides for an optimum spatial resolution only at this distance. If the radiation source is moved away from or towards the detector the strips are no longer pointing towards the radiation source and the spatial resolution is heavily deteriorated. The detector becomes practically useless if the distance is altered considerably.

A main object of the invention is therefore to provide a scanning-based ionizing radiation detecting apparatus and method, which provide for measurement of high spatial resolution independently of the distance from the detector to the radiation source used.

A further object of the invention is to provide such scanning-based ionizing radiation detecting apparatus and method, which provide a novel mechanism for increasing and decreasing the achievable signal-to-noise ratio, the spatial resolution and the detecting time of the detecting apparatus.

These objects, among others, are attained by apparatuses and methods as claimed in the appended claims.

By providing a scanning-based radiation detector apparatus for recording an image of an object comprising a one-dimensional detector exposed to a fan-shaped beam of ionizing radiation from a radiation source as transmitted through the object, the detector being of the kind wherein charges or photons generated by interactions between the fan-shaped radiation beam and a detection medium and traveling in a direction essentially perpendicular to the fan-shaped radiation beam; and a device for moving the detector and the fan-shaped radiation beam relative to the object while the detector is arranged to repeatedly detect to thereby create an image of the object, wherein the detector has a detecting arrangement for detecting the generated charges or photons, which comprises a large number of individual detecting elements, which can be grouped together to form a plurality of detecting stripes located side-by-side and pointing towards a selected single point, which coincides with the location of the radiation source, measurements of very high spatial resolution can be performed. The individual detecting elements are grouped together depending on the distance between the detector and the radiation source, and thus a single detector can be optimized for any given detector-radiation source distance.

Further, the plurality of detecting stripes, which are grouped together from the large number of individual detecting elements, may have a width corresponding to a selected required spatial resolution or signal-to-noise ratio. The narrower the stripes are, the better spatial resolution can be obtained, but to the cost of a deteriorated signal-to-noise ratio given a constant radiation flux. The wider the stripes are, the better signal-to-noise ratio can be obtained, but to the cost of a deteriorated spatial resolution. Still further, the plurality of detecting stripes, which are grouped together from the large number of individual detecting elements, are of a number, which corresponds to a selected required maximum detecting time given a detector such as a CCD-based detector, where the readout time can be affected by the grouping of individual detecting elements or pixels.

The one-dimensional detector is preferably, but not exclusively, a gaseous-based parallel plate detector. Other detectors that may be used include liquid-based parallel plate detectors, scintillator-based arrays, diode arrays, CCD arrays and other solid-state detectors with edge-on or near edge-on incidence of X-rays.

Further characteristics of the invention, and advantages thereof, will be evident from the detailed description of preferred embodiments of the present invention given hereinafter and the accompanying FIGS. 1-4, which are given by way of illustration only, and thus are not limitative of the present invention.

### BRIEF DESCRIPTION OF THE DRAWINGS

FIG. 1 illustrates schematically, in a side view, an apparatus for scanning-based X-ray imaging according to a preferred embodiment of the present invention.

FIG. 2 is a schematic enlarged cross-sectional view of some of the components of the apparatus of FIG. 1 taken along the line A-A of FIG. 1.

FIG. 3 is a schematic enlarged cross-sectional view of the same components of the apparatus of FIG. 1, but taken in an orthogonal direction as compared with the FIG. 2 view. Also a readout arrangement of a detector comprised in the apparatus of FIG. 1 is illustrated at a first operation setting.

FIG. 4a is a schematic enlarged top view of a readout arrangement of a detector comprised in the apparatus of FIG. 1 at a second operation setting.

FIG. 4b is a schematic enlarged top view of the readout arrangement of the detector comprised in the apparatus of FIG. 1 at a third operation setting.

### DETAILED DESCRIPTION OF PREFERRED EMBODIMENTS

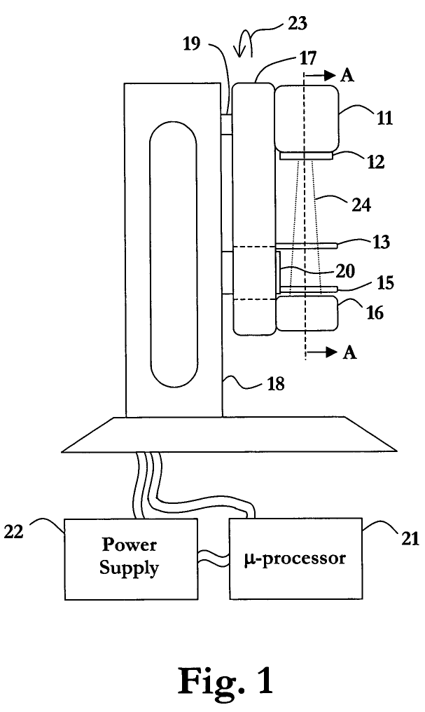

From top to bottom the apparatus in FIG. 1 comprises an X-ray source 11, a filter device 12, a fan beam collimator 13, an object table or holder 15, and a one-dimensional detector 16.

The X-ray source 11 is a conventional X-ray tube having a cathode, which emits electrons, and an anode emitting X-rays in response to being struck by said electrons. The resulting X-ray beam 24 is divergent.

The filter device 12 is located just beneath the X-ray tube 11, which typically includes thin metallic foils acting as filters to absorb the lowest and optionally also the highest energy photons, which do not contribute significantly to the image quality.

The fan beam collimator 13 may be a thin foil of e.g. tungsten with a narrow radiation transparent slit etched or cut away. The slit is aligned with a corresponding entrance of the detector 16 so that X-rays passing through the slit of the fan beam collimator 13 will enter the detector 16. However, due to the divergence of the radiation beam 24 the narrow radiation transparent slit has to be shorter and thinner than the entrance of the detector.

The detector 16, which will be described more in detail below, is a gaseous-based parallel plate detector arranged for repeated one-dimensional imaging of the radiation beam 24, being fan-shaped downstream of the collimator 13.

The X-ray tube 11, the fan beam collimator 13 and the detector 16 are attached to a common E-arm 17, which in turn is rotatably attached to a vertical stand 18 by means of a spindle 19 approximately at the height of the X-ray tube 11. In this manner, the X-ray tube 11, the fan beam collimator 13 and the detector 16 can be moved in a common pivoting movement relative to an examination object arranged on the object table 15 to scan the object and produce a two-dimensional image of the radiation transmitted through the object. The pivoting movement is schematically indicated by arrow 23.

The object table 15 is firmly attached to a support 20, which in turn is firmly attached to the vertical stand 18. For this purpose the E-arm 17 is provided with a recess or similar in the E-arm 17 (illustrated by the dashed lines). During scanning, the examination object (not illustrated) is located on the object table 15, which is kept still during scanning. Alternatively, the object is moved during scanning, while the X-ray tube 11, the fan beam collimator 13 and the detector 16 are kept at rest.

It shall be appreciated that the detector apparatus of FIG. 1 may be modified and arranged for linear movement of the X-ray tube 11, the fan beam collimator 13 and the detector 16 with respect to the object being examined. Yet alternatively, the fan beam collimator 13 and the detector 16 may be rotated 16 in the horizontal plane with respect to the object being examined.

Furthermore, the detector apparatus comprises a microprocessor or computer 21 provided with suitable software for controlling the apparatus and readout and post-processing of the signals from the line detector 16 and a power supply 22 for supplying the detector and the microprocessor or computer 21 with power and for driving a step motor or similar housed in the vertical stand 18 for driving the spindle 19 and thus the E-arm 17.

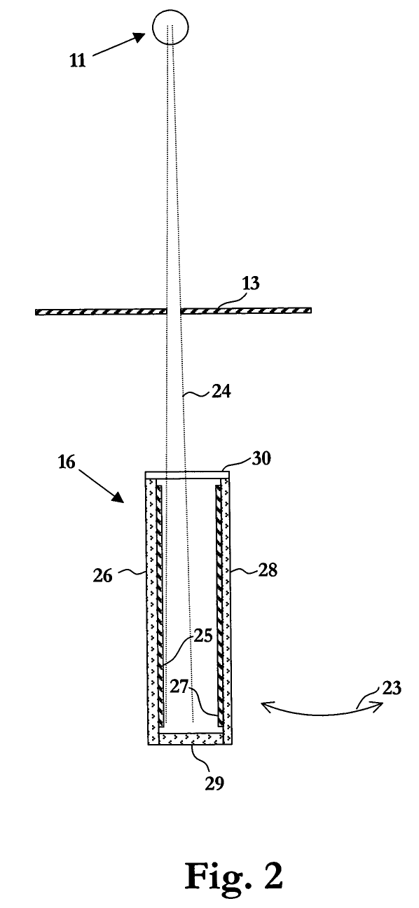

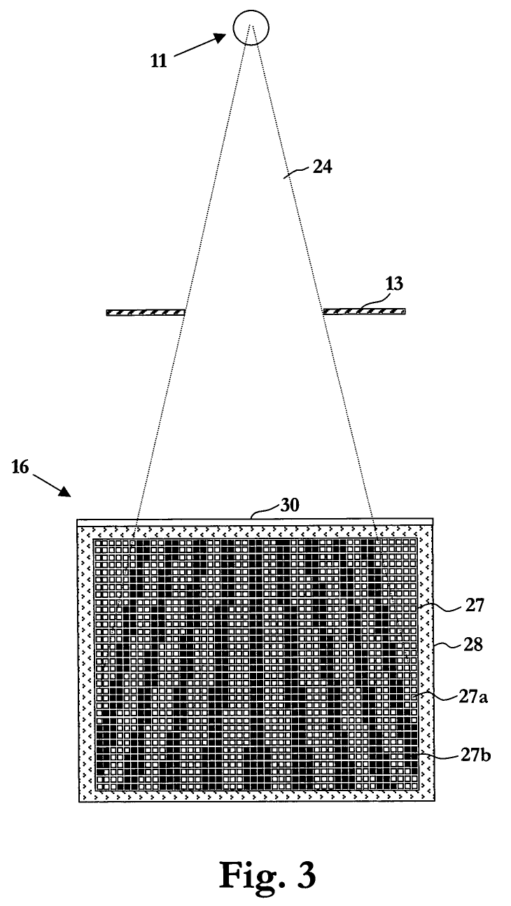

The detector 16 is illustrated more in detail in FIGS. 2-3 and is oriented such that the planar or fan-shaped X-ray beam 24 can enter sideways between essentially planar cathode 25, 26 and anode 27, 28 arrangements. Each of the electrode arrangements 25, 26; 27, 28 includes an electrically conducting electrode layer 25, 27 supported by a respective dielectric substrate 26, 28, wherein the arrangements are oriented so that the conductive cathode 25 and anode 27 layers are facing each other. A radiation transparent window 30 is provided at the front end of the detector to form an entrance for the fan-shaped beam 24 to the detector 16.

Preferably, the dielectric substrates 26, 28 and the window 30 define together with sidewalls 29 a gas-tight confinement capable of being filled with an ionizable gas or gas mixture. Alternatively, the electrode arrangements are arranged within an external gas-tight casing (not illustrated). The ionizable gas or gas mixture may e.g. comprise krypton and carbon dioxide or xenon and carbon dioxide.

The detector 16 comprises further a detecting or readout arrangement including a two-dimensional array of a large number of electrically insulated detecting or readout elements 27a as can be seen in FIG. 3. Typically, the anode arrangement 27, 28 constitutes the readout arrangement. Each of the readout elements 27a is preferably connected to the readout and signal-processing computer 21 as being illustrated in FIG. 1, whereupon the signals from each readout 27a can be processed separately.

Alternatively, the readout arrangement is arranged separately from the anode 25, but adjacent anode 27 or adjacent cathode 25, or elsewhere. The detector 16 may also comprise capabilities for electron avalanche amplification in order to record very low flux of X-rays, or detect each single X-ray with high efficiency.

The large number of individual detecting elements 27a are, according to the present invention, grouped together to form a plurality of detecting stripes or readout strips 27b side-by-side, where the stripes 27b are all pointing towards a selected single point, and each of them provides for a single signal value during detection. The grouping may be effectuated in the detector hardware or in the software of the computer 21 depending on the particular detector apparatus used.

In FIG. 3 seventeen such strips 27b are illustrated alternately using black and white filled readout elements for illustrative purposes (nine black strips and eight white strips interposed between the black ones). When the selected point now coincides with the location of the radiation source, any parallax errors in detected images are compensated for, thereby providing for an increased spatial resolution. The readout strips extend in directions parallel with the direction of incident photons of the X-ray beam 24 at each location. Given the divergent beam 24 from the radiation source 11 the readout strips 27b are arranged in a fan-like configuration. Further, for the same reason the readout elements 27a are grouped together in a manner so that the resulting readout strips are broader at the far end of the detector than at the front end thereof.

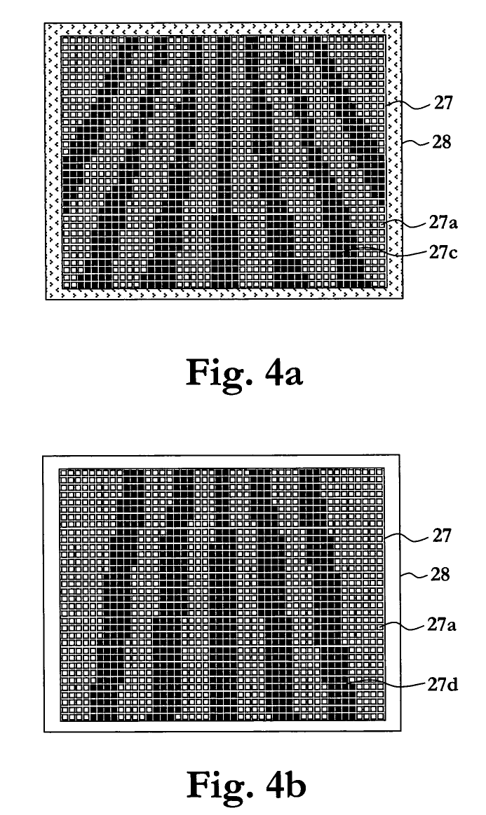

If the distance between the one-dimensional detector 16 and the radiation source 11 is altered, the large number of individual detecting elements 27a is just regrouped to fit that distance. For instance, the readout arrangement is illustrated in FIG. 4a as being used with a radiation source located closer to the detector. Here, seven black strips and six white strips interposed between the black ones are illustrated. As can be seen they are considerably less parallel than the strips of FIG. 3.

It shall be understood that while the illustrated detector has a limited number of readout elements 27a for sake of simplicity, a preferred detector may comprise at least 1000, preferably at least 10000, more preferably at least 100000, and most preferably at least 1000000, individual readout elements 27a. Each of the readout elements 27a may have a quadratic or rectangular shape and a detecting area, which measures less than 1 mm², preferably less than 0.25 mm², and more preferably less than 0.01 mm², and most preferably less than 0.0025 mm². Thus, the number of readout strips 27b obtained by grouping may be much larger than what is illustrated and the strips will appear with much smoother delimitation. A spatial resolution better than 0.1 mm is easily achievable. If the readout elements 27a are rectangular they are preferably arranged so that their longer sides are parallel with the central axis of the radiation beam 24. Alternatively, each of the readout elements 27a may have a trapezium shape and the readout elements in the far end of the detector may be broader than the readout elements in the front end thereof.

Further, the plurality of detecting stripes, which are grouped together from the large number of individual detecting elements, may have a width corresponding to a selected required spatial resolution or signal-to-noise ratio. The narrower the stripes are, the better spatial resolution can be obtained, but to the cost of a deteriorated signal-to-noise ratio for a given X-ray flux. The wider the stripes are, the better signal-to-noise ratio is obtainable, but to the cost of a deteriorated spatial resolution. In FIG. 4b the readout arrangement is illustrated as being used in a mode with higher signal-to-noise ratio and lower spatial resolution. Here, five black strips and four white strips interposed between the black ones are illustrated. As can be seen they are considerably broader than the strips of FIG. 3. Thus, given a selected required signal-to-noise ratio for a certain radiation flux or a selected spatial resolution, the required width of the strips to be used can be calculated, and the readout elements 27a may be grouped accordingly.

As the number of strips is reduced, the speed of the detector can be improved depending on the readout mechanism of the detector. Thus, given a selected required maximum detecting time, the maximum number of strips to be used can be calculated, and the readout elements 27a may be grouped accordingly.

In an alternative version of the apparatus of FIG. 1, the detector is replaced by a detector arrangement comprising a plurality of the inventive one-dimensional detector distributed in a one- or two-dimensional array. The fan beam collimator 13 is replaced by a collimator with a plurality of narrow radiation transparent slits—one for each detector. The dimensions and orientations of the slits are such that each detector is exposed to a respective fan-shaped X-ray beam. By such version the scanning distance and time can be considerably shortened.

For further details regarding different kind of detectors for use in the present invention, reference is made to the following U.S. Patents by Tom Francke et al. and assigned to XCounter AB of Sweden, which patents are hereby incorporated by reference: U.S. Pat. Nos. 6,118,125; 6,373,065; 6,337,482; 6,385,282; 6,414,317; 6,476,397; 6,477,223; 6,518,578; 6,522,722; 6,546,070; 6,556,650; 6,600,804; and 6,627,897.

It shall further be appreciated that while the detector in the description above has been described as a gaseous-based ionization detector, wherein the freed electrons are drifted in a direction essentially perpendicular to the direction of the incident ionization, the present invention is not limited to such a detector. Examples of detectors that can be used in the present invention include liquid-based parallel plate detectors, edge-on scintillator-based arrays, diode arrays and other solid-state detectors with edge-on or near edge-on incidence of X-rays.

1. A scanning-based radiation detector apparatus for recording an image of an object comprising:
a one-dimensional detector exposed to a fan-shaped beam of ionizing radiation from a radiation source as transmitted through said object, and arranged for repeated one-dimensional imaging of said fan-shaped beam of ionizing radiation, said one-dimensional detector being of the kind wherein charges or photons generated by interactions between said fan-shaped beam of ionizing radiation and a detection medium and traveling in a direction essentially perpendicular to said fan-shaped beam of ionizing radiation, are detected; and
a device for moving said one-dimensional detector and said fan-shaped beam of ionizing radiation relative to said object while said one-dimensional detector is arranged to repeatedly detect to thereby create an image of the object, wherein
said one-dimensional detector has a detecting arrangement for detecting said charges or photons, which comprises a large number of individual detecting elements, and means for grouping said large number of individual detecting elements together to form a plurality of detecting stripes side-by-side, which plurality of detecting stripes are all pointing towards a selected single point.

2. The scanning-based radiation detector apparatus of claim 1 wherein said selected single point coincides with the location of said radiation source.

3. The scanning-based radiation detector apparatus of claim 1 wherein said means for grouping is arranged to regroup said large number of individual detecting elements if the distance between the one-dimensional detector and the radiation source is altered.

4. The scanning-based radiation detector apparatus of claim 1 wherein said means for grouping is arranged to group said large number of individual detecting elements together to form a plurality of detecting stripes, which each has a width corresponding to a selected required spatial resolution.

5. The scanning-based radiation detector apparatus of claim 1 wherein said means for grouping is arranged to group said large number of individual detecting elements together to form a plurality of detecting stripes, which each has a width corresponding to a selected required signal-to-noise ratio.

6. The scanning-based radiation detector apparatus of claim 1 wherein said means for grouping is arranged to group said large number of individual detecting elements together to form a plurality of detecting stripes, which is of a number, which corresponds to a selected required maximum detecting time.

7. The scanning-based radiation detector apparatus of claim 1 wherein said means for grouping is arranged to group said large number of individual detecting elements together to form a plurality of detecting stripes, which each has a varying width.

8. The scanning-based radiation detector apparatus of claim 1 wherein said large number of individual detecting elements is distributed in a two-dimensional array.

9. The scanning-based radiation detector apparatus of claim 1 wherein said large number is at least 1000.

10. The scanning-based radiation detector apparatus of claim 1 wherein said large number is at least 10000.

11. The scanning-based radiation detector apparatus of claim 1 wherein said large number is at least 100000.

12. The scanning-based radiation detector apparatus of claim 1 wherein said large number is at least 1000000.

13. The scanning-based radiation detector apparatus of claim 1 wherein the detecting area of each of said large number of individual detecting elements measures less than 1 mm².

14. The scanning-based radiation detector apparatus of claim 1 wherein the detecting area of each of said large number of individual detecting elements measures less than 0.25 mm².

15. The scanning-based radiation detector apparatus of claim 1 wherein the detecting area of each of said large number of individual detecting elements measures less than 0.01 mm².

16. The scanning-based radiation detector apparatus of claim 1 wherein the detecting area of each of said large number of individual detecting elements measures less than 0.0025 mm².

17. The scanning-based radiation detector apparatus of claim 1 wherein said one-dimensional detector is any of a gaseous-based parallel plate detector, liquid-based parallel plate detector, a scintillator-based array, a diode array or a solid-state detector.

18. A method for recording an image of an object comprising the steps of:
indicating a distance between a radiation source of ionizing radiation and a one-dimensional detector, wherein the one-dimensional detector is of the kind wherein charges or photons generated by interactions between an incident radiation beam of ionizing radiation and a detection medium and traveling in a direction essentially perpendicular to the radiation beam of ionizing radiation, are detected; and has a detecting arrangement for detecting said charges or photons, which comprises a large number of individual detecting elements;
grouping said large number of individual detecting elements together to form a plurality of detecting stripes side-by-side depending on said indicated distance;
exposing said one-dimensional detector to a fan-shaped beam of ionizing radiation from said radiation source as transmitted through said object; and
moving said one-dimensional detector and said fan-shaped beam of ionizing radiation relative to said object while said repeatedly detecting by said one-dimensional detector to thereby create an image of the object.

19. The method of claim 18 wherein said large number of individual detecting elements are grouped together to form said plurality of detecting stripes to all point towards said source of ionizing radiation.

20. The method of claim 18 wherein said method is repeated if the distance between the one-dimensional detector and the radiation source is altered.

21. The method of claim 18 wherein
a required spatial resolution or signal-to-noise ratio is indicated; and
said large number of individual detecting elements are grouped together to form each of said plurality of detecting stripes with a width depending on said indicated required spatial resolution.

22. The method of claim 18 wherein
a required spatial resolution or signal-to-noise ratio is indicated; and
said large number of individual detecting elements are grouped together to form each of said plurality of detecting stripes with a width depending on said indicated required signal-to-noise ratio.

23. The method of claim 18 wherein
a required maximum detecting time is indicated; and
said large number of individual detecting elements are grouped together to form said plurality of detecting stripes in a number, which depends on said indicated required maximum detecting time.

24. The method of claim 18 wherein said large number of individual detecting elements are grouped together to form each of said plurality of detecting stripes with a varying width.
